(19) United States
(12) Patent Application Publication
(10) Pub. No.: US 2026/0240954 A1
(43) Pub. Date: Aug. 20, 2026

(54) PHARMACEUTICAL COMPOSITION COMPRISING LIQUID AND MICROSPHERE FORMULATIONS, WHICH COMPRISE ONE OR MORE SELECTED FROM GROUP CONSISTING OF GLP-1 RECEPTOR AGONIST, GIP/GLP-1 RECEPTOR DUAL AGONIST AND GLP-1/GIP/GCG RECEPTOR TRIPLE AGONIST

(71) Applicant: AULBIO CO., LTD., Seongnam-si, Gyeonggi-do (KR)

(72) Inventors: Cherng Ju KIM, Beaumont, CA (US); Tae Kun AN, Yongin-si (KR); A Ram KIM, Seoul (KR); Seo A KIM, Gyeonggi-do (KR); Ji Won PARK, Gyeonggi-do (KR); Gyu Ri KIM, Gyeonggi-do (KR); Do Hee KIM, Suwon-si (KR)

(73) Assignee: AULBIO CO., LTD., Seongnam-si, Gyeonggi-do (KR)

(21) Appl. No.: 19/471,903

(22) PCT Filed: Apr. 5, 2024
(86) PCT No.: PCT/KR2024/004565

§ 371 Date: Oct. 2, 2025

(30) Foreign Application Priority Data
Apr. 5, 2023 (KR) 10-2023-0044574
Apr. 5, 2024 (KR) 10-2024-0046706

(51) Int. Cl. A61K 38/26; A61K 9/00; A61K 9/08; A61K 9/16; A61K 47/10

(52) U.S. Cl. CPC A61K 38/26; A61K 9/0019; A61K 9/08; A61K 9/1694; A61K 47/10

(57) ABSTRACT

The present invention provides a pharmaceutical composition comprising: as a first active ingredient, at least one selected from the group consisting of a GLP-1 receptor agonist, a GIP/GLP-1 receptor dual agonist, and a GLP-1/GIP/GCG receptor triple agonist or a pharmaceutically acceptable salt thereof; as a second active ingredient, a microsphere containing at least one selected from the group consisting of a GLP-1 receptor agonist, a GIP/GLP-1 receptor dual agonist, and a GLP-1/GIP/GCG receptor triple agonist or a pharmaceutically acceptable salt thereof; and a pharmaceutically acceptable liquid carrier.

### TECHNICAL FIELD

This application claims the benefit of priority to Korean Patent Application No. 10-2023-0044574 filed on Apr. 5, 2023, and Korean Patent Application No. 10-2024-0046706 filed on Apr. 5, 2024, the entire contents of which are incorporated as part of this specification.

The present invention relates to a pharmaceutical composition comprising liquid and microsphere formulations, which comprise one or more selected from the group consisting of GLP-1 receptor agonist, GIP/GLP-1 receptor dual agonist, and GLP-1/GIP/GCG receptor triple agonist.

### BACKGROUND ART

Glucagon-like peptide-1 (GLP-1) is derived from pre-proglucagon, a 158 amino acid precursor polypeptide that is processed in different tissues to form numerous different proglucagon-derived peptides, including glucagon, glucagon-like peptide-1 (GLP-1), glucagon-like peptide-2 (GLP-2), and oxyntomodulin (OXM), which are involved in the regulation of food intake as well as various physiological functions, including glucose homeostasis, insulin secretion, gastric emptying, and intestinal growth.

GLP-1 is produced as a 37-amino acid peptide corresponding to amino acids 72-108 of proglucagon (amino acids 92-128 of preproglucagon). GLP-1 (7-36) amide or GLP-1 (7-37) acid is the biologically active form of GLP-1, which exhibits essentially identical activity at the GLP-1 receptor. GLP-1 and GLP-1 analogues, which act as agonists at the GLP-1 receptor, have been shown to provide, for example, effective glycemic control for the treatment of patients with type 2 diabetes, and in addition, to provide weight loss, preservation of beta-cell function, and alleviation of hypertension, hypoglycemia, and/or hyperlipidemia.

Currently, certain GLP-1 analogues are marketed or under development, including exenatide, semaglutide, liraglutide, lixisenatide, albiglutide, and dulaglutide.

Additionally, Mounjaro® (tirzepatide), which acts not only on GLP-1 but also on GIP (Gastric Inhibitory polypeptide) receptors, is commercially available.

Like GLP-1, GIP increases insulin secretion by increasing the production and inhibiting the destruction of insulin-secreting beta cells in the pancreas. It reduces fat accumulation, promotes bone formation, and increases glucagon production while reducing gastric juice secretion.

Additionally, retatrutide is known to act as a GLP-1/GIP/GCG receptor triple agonist, and the GCG receptor regulates the action of glucagon in the liver. Glucagon promotes gluconeogenesis in the liver to increase blood sugar levels, and GCG receptors inhibit the action of glucagon to maintain a constant blood sugar level.

A GLP-1 receptor agonist, a GIP/GLP-1 receptor dual agonist, or a GLP-1/GIP/GCG receptor triple agonist is mainly administered by injection due to several barriers such as enzymatic degradation in the gastrointestinal tract and intestinal mucosa, insufficient absorption from the intestinal mucosa, and first pass metabolism in the liver. Recently, oral administration has also been commercialized, but its bioavailability is very low compared to the injection, so the dosage is considerably high.

GLP-1 receptor agonist, GIP/GLP-1 receptor dual agonist, or GLP-1/GIP/GCG receptor triple agonist injections are manufactured in liquid formulation. While these liquid formulations are easy to manufacture and administer, increasing the dosage can cause fatal side effects. Therefore, their use for maintaining long-term efficacy is limited.

Recently, to overcome these shortcomings, microsphere formulations using biodegradable polymers have been developed, and it is possible to design them to exhibit long-term effects. However, due to the nature of peptides, it is difficult to secure bioavailability.

### PRIOR ART DOCUMENT

### Patent Document

Korean Laid-open Patent Publication No. 10-2020-0044016.

### DISCLOSURE

### Technical Problem

The present invention has been devised to solve the above-described problems of the prior art, and aims to provide a pharmaceutical composition with improved bioavailability by mixing and using a liquid formulation containing at least one selected from the group consisting of a GLP-1 receptor agonist, a GIP/GLP-1 receptor dual agonist, and a GLP-1/GIP/GCG receptor triple agonist and a microsphere formulation.

### Technical Solution

To solve the above problem, the present invention provides a pharmaceutical composition comprising:
as a first active ingredient, at least one selected from the group consisting of a GLP-1 receptor agonist, a GIP/GLP-1 receptor dual agonist, and a GLP-1/GIP/GCG receptor triple agonist, or a pharmaceutically acceptable salt thereof,
as a second active ingredient, a microsphere containing at least one selected from the group consisting of a GLP-1 receptor agonist, a GIP/GLP-1 receptor dual agonist, and a GLP-1/GIP/GCG receptor triple agonist, or a pharmaceutically acceptable salt thereof, and
a pharmaceutically acceptable liquid carrier.

### Advantageous Effects

The pharmaceutical composition of the present invention provides improved bioavailability by mixing a liquid formulation containing at least one selected from the group consisting of a GLP-1 receptor agonist, a GIP/GLP-1 receptor dual agonist, and a GLP-1/GIP/GCG receptor triple agonist and a microsphere formulation.

### BEST MODE

Hereinafter, the present invention will now be described in more detail.

All technical terms used herein, unless otherwise defined, have the same meaning as commonly understood by those skilled in the art. Furthermore, methods or samples similar or equivalent to those described as preferred methods or samples herein are also included within the scope of the present invention. The contents of all publications cited as references herein are hereby incorporated by reference in their entirety.

The present invention relates to a pharmaceutical composition comprising:
as a first active ingredient, at least one selected from the group consisting of a GLP-1 receptor agonist, a GIP/GLP-1 receptor dual agonist, and a GLP-1/GIP/GCG receptor triple agonist, or a pharmaceutically acceptable salt thereof,
as a second active ingredient, a microsphere containing at least one selected from the group consisting of a GLP-1 receptor agonist, a GIP/GLP-1 receptor dual agonist, and a GLP-1/GIP/GCG receptor triple agonist, or a pharmaceutically acceptable salt thereof, and
a pharmaceutically acceptable liquid carrier.

Since the first active ingredient and the liquid carrier form a liquid formulation, and the microsphere containing the second active ingredient forms a microsphere formulation, the pharmaceutical composition may also be expressed as follows.

A pharmaceutical composition comprising a liquid formulation comprising: as a first active ingredient, at least one selected from the group consisting of a GLP-1 receptor agonist, a GIP/GLP-1 receptor dual agonist, and a GLP-1/GIP/GCG receptor triple agonist, or a pharmaceutically acceptable salt thereof, and
a microsphere formulation comprising, as a second active ingredient, at least one selected from the group consisting of a GLP-1 receptor agonist, a GIP/GLP-1 receptor dual agonist, and a GLP-1/GIP/GCG receptor triple agonist, or a pharmaceutically acceptable salt thereof.

At this time, the liquid formulation may further comprise a pharmaceutically acceptable liquid carrier.

Improving the ease of dosing and safety of GLP-1 receptor agonists, GIP/GLP-1 receptor dual agonists, or GLP-1/GIP/GCG receptor triple agonists are constant challenges facing researchers in this field. Therefore, researchers have proposed various solutions to address these challenges. However, no previous research has proposed the idea of using by mixing liquid and microsphere formulations.

The present invention is characterized in that bioavailability is significantly improved by using a mixture of a liquid formulation and a microsphere formulation as described above.

In one embodiment of the present invention, the pharmaceutically acceptable salt for the first active ingredient or the second active ingredient may be, but is not limited to, sodium, ammonium, acetic acid, benzoic acid, potassium, magnesium, calcium, hydroxynaphthoic acid, napadisylate, pamoate, fumaric acid, oxalic acid, citric acid, tartaric acid, hydrochloric, phosphoric acid, maleic acid, mesylic acid, edisylic acid, succinic acid, aspartic acid, pamoic acid, sulfuric acid, besylic acid, or tosylic acid salts of at least one selected from the group consisting of a GLP-1 receptor agonist, a GIP/GLP-1 receptor dual agonist, and a GLP-1/GIP/GCG receptor triple agonist.

In one embodiment of the present invention, as the first active ingredient or the second active ingredient, at least one selected from the group consisting of a GLP-1 receptor agonist, a GIP/GLP-1 receptor dual agonist and a GLP-1/GIP/GCG receptor triple agonist may be at least one selected from the group consisting of exenatide, liraglutide, lixisenatide, albiglutide, dulaglutide, semaglutide, tirzepatide, cotadutide, taspoglutide, and retatrutide.

The first active ingredient and the second active ingredient may be selected identically or differently.

In one embodiment of the present invention, the second active ingredient included in the microsphere may be included in an amount 3 to 1000 times the weight of the first active ingredient.

Specifically, the weight of the second active ingredient included in the microsphere may be included at 3 times or more, 5 times or more, 6 times or more, 7 times or more, 8 times or more, 9 times or more, 10 times or more, 11 times or more, or 12 times or more than the weight of the first active ingredient, and may be included at 1000 times or less, 900 times or less, 800 times or less, 700 times or less, 600 times or less, 500 times or less, 400 times or less, 300 times or less, or 200 times or less, 150 times or less, 100 times or less, or 50 times or less than the weight of the first active ingredient. Additionally, it may also be included in a range consisting of one lower limit and one upper limit described above.

In the above, if the weight of the second active ingredient included in the microsphere is less than 3 times, it is not desirable because it cannot exert long-term efficacy with a single administration, and if it is included in excess of 1000 times, it is not desirable because the side effects due to the excessive amount of drug included in the microsphere may be greater than the improvement in convenience of administration.

In one embodiment of the present invention, the first active ingredient and the second active ingredient may be contained in a total amount of 0.001 mg to 2,000 mg per 1 mL of the liquid carrier. The liquid carrier may be, for example, a carrier for injection.

In one embodiment of the present invention, the liquid carrier may include, but is not limited to, at least one selected from the group consisting of water, surfactants, and polyhydric alcohols and a pharmaceutically acceptable excipient. That is, the liquid carrier may include solvents, excipients, etc. that are commonly used in injections in this field, and at this time, the solvents, excipients, etc. may be included in an amount known in this field.

The surfactant may include at least one selected from, but is not limited to, Poloxamer 188, Poloxamer 407, Polysorbate 20, Polysorbate 60, Polysorbate 80, Polyoxyethylene oleyl ether, Polyoxyethylene cetyl ether, Polyoxyethylene stearyl ether, and Polyoxyethylene lauryl ether.

The polyhydric alcohol may include, but is not limited to, at least one selected from ethylene glycol, propylene glycol, polyethylene glycol, glycerol, erythritol, threitol, xylitol, ribitol, mannitol, sorbitol, and maltitol.

The excipients may include, but are not limited to, buffers, isotonic agents, preservatives, and pH regulators, for example, and excipients known in the art may be used without limitation.

As the buffer, for example, phosphate buffer, TRIS, citrate, etc. can be used, but are not limited thereto, and buffers known in the art can be used without limitation. The phosphate buffer can include sodium dihydrogen phosphate, disodium hydrogen phosphate, sodium phosphate, etc.

The buffer may be included at a concentration of, for example, 0.01 to 50 mM.

As the isotonic agent, for example, propylene glycol, sodium chloride, mannitol, sucrose, sorbitol, trehalose, lactose, etc. can be used, but are not limited thereto, and any isotonic agent known in the art may be used without limitation. The isotonic agent may be included in the liquid formulation at a concentration of 8 mg/ml to 50 mg/ml, for example, at a concentration of 8 mg/ml to 30 mg/ml.

As the preservative, for example, phenol may be used, but is not limited thereto, and any preservative known in the art may be used without limitation.

As the pH regulator, for example, HCl, NaOH, or acetate may be used, but is not limited thereto, and any pH regulator known in the art may be used without limitation.

The pH of the liquid carrier can be adjusted to a range of 3 to 10, 5 to 9, or 5.5 to 8.5.

In one embodiment of the present invention, the microsphere containing the second active ingredient may further comprise a biodegradable polymer.

In one embodiment of the present invention, the biodegradable polymer may be at least one selected from, for example, polylactic acid, polylactide, polylactic-co-glycolic acid, polylactide-co-glycolide (PLGA), polyphosphazine, polyiminocarbonate, polyphosphoester, polyanhydride, polyorthoester, copolymer of lactic acid and caprolactone, polycaprolactone, polyhydroxyvalerate, polyhydroxybutyrate, polyamino acid, and copolymer of lactic acid and amino acid.

In one embodiment of the present invention, the microsphere may include one or more biodegradable polymers.

In one embodiment of the present invention, the biodegradable polymer having an intrinsic viscosity of 0.15 dL/g to 1.7 dL/g, 0.15 dl/g to 1.3 dL/g, or 0.15 dl/g to 1.2 dL/g may be used.

The intrinsic viscosity means that measured at a concentration of 0.05% (w/v) to 3% (w/v) in chloroform at 25° C. using an Ubbelohde viscometer. When the intrinsic viscosity is less than 0.15 dL/g, the molecular weight of the polymer is insufficient, making it difficult to exhibit a sustained-release effect of at least one selected from a GLP-1 receptor agonist, a GIP/GLP-1 receptor dual agonist, a GLP-1/GIP/GCG receptor triple agonist and a pharmaceutically acceptable salts thereof. When the intrinsic viscosity exceeds 1.7 dL/g, an effect of excessively delaying the release of at least one selected from a GLP-1 receptor agonist, a GIP/GLP-1 receptor dual agonist, a GLP-1/GIP/GCG receptor triple agonist and a pharmaceutically acceptable salts thereof may occur. In addition, when manufacturing microspheres using a polymer with high intrinsic viscosity, there is a problem in that an excessive amount of manufacturing solvent must be used due to the high viscosity of the polymer, and it is difficult to manufacture reproducible microspheres.

Examples of commercially available polymers having the above properties include Evonik company's Resomer series RG502H, RG503H, RG504H, RG502, RG503, RG504, RG653H, RG752H, RG752S, RG755S, RG750S, RG757S, RG858S, R202H, R203H, R205H, R202S, R203S, R205S, R206S and R207S, and Corbion company's PDL 02A, PDL 02, PDL 04, PDL 05, PDLG 7502A, PDLG 7502, PDLG 7507, PDLG 5002A, PDLG 5002, PDLG 5004A, and PDLG 5004, etc.

In one embodiment of the present invention, the microsphere may further include at least one release-controlling agent selected from a surfactant, a polyhydric alcohol, butyric acid, valeric acid, caproic acid, enanthic acid, caprylic acid, pelargonic acid, capric acid, undecylic acid, lauric acid, tridecylic acid, myristic acid, pentadecylic acid, palmitic acid, heptadecylic acid, stearic acid, nonadecylic acid, arachidic acid, isocrotonic acid, oleic acid, elaidic acid, sorbic acid, linoleic acid, arachidonic acid, hydroxynaphthoic acid, napadiylic acid, and pamoic acid.

In one embodiment of the present invention, it may be more preferable that the second active ingredient and the biodegradable polymer included in the microsphere are included in a weight ratio of 1:1 to 50, 1:1.5 to 50, 1:1.6 to 50, 1:1.7 to 50, 1:1 to 47, 1:1.5 to 47, 1:1.6 to 47, 1:1.7 to 47, 1:1 to 45, 1:1.5 to 45, 1:1.6 to 45, 1:1.7 to 45, 1:1 to 42, 1:1.5 to 42, 1:1.6 to 42, 1:1.7 to 42, 1:1 to 40, 1:1.5 to 40, 1:1.6 to 40, or 1:1.7 to 40.

At this time, if the weight ratio of the biodegradable polymer to the second active ingredient is less than 1, it is not desirable because it may cause fatal side effects due to excessive release of the initial active ingredient, and if the weight ratio exceeds 50, the dissolution of the second active ingredient becomes too slow, making it difficult to secure a constant blood drug profile.

The content range of the second active ingredient included in the microsphere may be 1 to 50 wt %, 2 to 45 wt %, or 3 to 40 wt % with respect to the total weight of the microsphere.

Additionally, the content range of the biodegradable polymer contained in the microsphere may be 50 to 99 wt %, 55 to 98 wt %, or 60 to 97 wt % with respect to the total weight of the microsphere.

In one embodiment of the present invention, the microsphere may have a release rate of the second active ingredient within 7 days of 60 wt % or less or 55 wt % or less.

Additionally, the microsphere may have a release rate of the second active ingredient within 3 days of 45 wt % or less or 40 wt % or less.

In particular, it is preferable that the second active ingredient release rate in the microsphere of the present invention during the initial day is 30 wt % or less and 25 wt % or less. In addition, it is preferable that the microsphere of the present invention continuously releases the drug for 1 month or more, 2 months or more, 3 months or more, 4 months or more, 5 months or more, or 6 months or more after administration, except for the initial administration period.

In one embodiment of the present invention, the pharmaceutical composition may be in the form of an injection.

In one embodiment of the present invention, the pharmaceutical composition may have a form in which microspheres are dispersed in a liquid formulation prepared by dissolving or dispersing the first active ingredient in a liquid carrier. For example, the pharmaceutical composition may have a form in which microspheres are dispersed within an injection, which is a liquid formulation.

Additionally, in one embodiment of the present invention, the pharmaceutical composition may have a form in which a liquid carrier is added to a microsphere containing the first active ingredient and the second active ingredient. As the liquid carrier, for example, an injectable carrier (or solvent) can be used.

In one embodiment of the present invention, the pharmaceutical composition may be administered to a subject at intervals of 1 to 6 months.

In one embodiment of the present invention, the pharmaceutical composition can be used for the prevention or treatment of diabetes, hypertension, hyperlipidemia, obesity, non-alcoholic steatohepatitis, metabolic steatohepatitis, cardiovascular disease, or degenerative neurological diseases such as Alzheimer's disease and Parkinson's disease. The pharmaceutical composition of the present invention can be formulated into various forms of preparations, for example, it can be formulated into a parenteral administration preparation.

The pharmaceutical composition of the present invention can be formulated for parenteral administration, and can be formulated in the form of, for example, injections, creams, lotions, ointments for external use, oils, moisturizers, gels, aerosols, patches, and nasal inhalers, but is not limited thereto.

The parenteral administration formulation may further include a thickener, a stabilizer, an isotonic agent, a pH regulator, a surfactant, an excipient and/or a carrier in addition to the microsphere containing the first active ingredient and the second active ingredient.

The usable isotonic agents include water-soluble excipients or sugars such as mannitol, sucrose, sorbitol, trehalose, lactose, and sodium chloride.

Examples of the thickener include sodium carmellose, sodium carboxymethyl cellulose, povidone, etc.

Examples of the surfactants include polyvinyl alcohol, etc. In addition, examples of buffering agents include sodium monohydrogen phosphate, anhydrous citric acid, sodium hydroxide, sodium chloride, etc.

The parenteral administration formulation is not limited to the forms exemplified above and may be formulated in a manner known in the art.

The pharmaceutical composition of the present invention can be administered in a therapeutically effective amount of the first and second active ingredients, for example, an amount effective for treating diabetes, specifically type 2 diabetes, preservation of beta-cell function, hypertension, hyperlipidemia, obesity, non-alcoholic steatohepatitis, metabolic steatohepatitis, cardiovascular disease, or degenerative neurological diseases such as Alzheimer's disease and Parkinson's disease. The therapeutically effective amount of the active ingredient can be evaluated by a physician.

The pharmaceutical composition of the present invention may be administered once every two weeks, once every three weeks, once every month, once every three months, once every six months, or once every year, but is not limited thereto.

The pharmaceutical composition of the present invention can be administered, for example, via subcutaneous injection. The subcutaneous injection may be performed, for example, using a pre-filled syringe, for example, a pen-syringe, which may be a disposable syringe containing a single dose, or a metered dose syringe (syringe with dose metering device) in which only a single dose is injected at a time.

The pharmaceutical composition of the present invention exhibits sufficient efficacy as a GLP-1 receptor agonist and/or a GIP/GLP-1 receptor dual agonist and/or a GLP-1/GIP/GCG receptor triple agonist during the treatment period, and can be usefully used for the prevention or treatment of diabetes, specifically type 2 diabetes, preservation of beta-cell function, hypertension, hyperlipidemia, obesity, non-alcoholic steatohepatitis, metabolic steatohepatitis, cardiovascular disease, metabolic steatohepatitis, cardiovascular disease, or degenerative neurological diseases such as Alzheimer's disease and Parkinson's disease.

The pharmaceutical composition of the present invention can be prepared by preparing a pharmaceutically acceptable liquid carrier and mixing therein the first active ingredient and a microsphere containing the second active ingredient prepared in advance. The pharmaceutical composition can be prepared so that the total active ingredient is contained in an amount of 0.0001 to 200 parts by weight, or 0.001 to 200 parts by weight, based on 100 parts by weight of the liquid carrier.

The microsphere included in the pharmaceutical composition of the present invention can be prepared using a method known in the art, for example, a “solvent extraction and evaporation method”.

Specifically, it can be manufactured by a method comprising the following steps:
(a) preparing a solution (dispersed phase) containing the active ingredient and the polymer by dissolving the second active ingredient and the biodegradable polymer in an organic solvent;
(b) preparing an emulsion by adding the second active ingredient and the polymer-containing solution prepared in the above step (a) to an aqueous phase (continuous phase);
(c) forming a microsphere by extracting and evaporating the organic solvent from the dispersed phase of the emulsion prepared in the above step (b) into the continuous phase; and
(d) recovering the microsphere from the continuous phase of the above step (c) to prepare a microsphere containing the second active ingredient.

In the above manufacturing method, the second active ingredient and biodegradable polymer used have been described above, so their description is omitted here.

In the above step (a), the second active ingredient can be first dissolved in distilled water and then mixed with the biodegradable polymer solution.

In the above step (a), an organic solvent that is miscible with water or an organic solvent that is immiscible with water may be used as the organic solvent. Alternatively, a mixed solvent of an organic solvent that is miscible with water and an organic solvent that is immiscible with water may be used.

It is preferable to use the organic solvent having a property of being immiscible with water in an amount of 50% (v/v) or more of the total solvent. When utilizing the property of being immiscible with water of the organic solvent, an emulsion can be efficiently formed by homogeneously mixing the dispersed phase in the continuous phase.

The type of organic solvent that dissolves the second active ingredient and one or more biodegradable polymers is not particularly limited, but preferably, one or a mixture of two or more solvents selected from the group consisting of dichloromethane, chloroform, ethyl acetate, methyl ethyl ketone, acetone, acetonitrile, dimethyl sulfoxide, dimethyl formamide, N-methyl pyrrolidone, acetic acid, methyl alcohol, ethyl alcohol, propyl alcohol, and benzyl alcohol may be used.

In the above step (b), the step of preparing an emulsion by adding the second active ingredient and the polymer-containing solution (dispersed phase) to the aqueous phase (continuous phase) can be performed using a homogenizer such as a high-speed stirrer, an inline mixer, a membrane emulsion method, a microfluidic emulsion method, an ultrasonic mixer, or a static mixer.

In the above step (b), the continuous phase may further include a surfactant. The type of the surfactant is not particularly limited, and any surfactant that can help the second active ingredient and the polymer solution to form a stable droplet dispersion phase within the continuous phase may be used. As the surfactant, a hydrophilic polymer selected from the group consisting of methylcellulose, polyvinylpyrrolidone, carboxymethylcellulose, lecithin, gelatin, polyvinyl alcohol, polyoxyethylene-polyoxypropylene block copolymer, polyoxyethylene sorbitan fatty acid ester, polyoxyethylene castor oil derivative, and mixtures thereof can be used, and polyvinyl alcohol can be particularly preferably used.

The surfactant may be added to the continuous phase at a concentration of 0.1 to 3% (w/v), more preferably 0.2 to 3% (w/v). If the concentration of the surfactant is less than 0.1%, a dispersed phase or emulsion in the form of droplets may not be formed within the continuous phase, and if the concentration of the surfactant exceeds 3%, it may be difficult to remove the surfactant after the microspheres are formed.

Water can be used as the aqueous solution (continuous phase) of the above step (b), and the pH of the aqueous solution can be 4.0 to 10, but is not limited thereto. NaCl, which acts as an osmotic regulator, is used to control the initial release.

In the above step (c), if the emulsion is maintained or stirred for a certain period of time, for example, 2 to 48 hours, the organic solvent can be extracted from the dispersed phase into the continuous phase. A portion of the organic solvent extracted into the continuous phase can evaporate from the surface. At this time, the dispersed phase in the form of droplets can solidify to form microspheres.

In order to efficiently remove the organic solvent in the above step (c), the continuous phase may be heated for a certain period of time. The heating temperature is not limited and can be appropriately adjusted by a person skilled in the art depending on the organic solvent used.

The method of recovering microspheres in the above step (d) can be performed using various known techniques, for example, methods such as filtration or centrifugation can be used.

Between the above steps (c) and (d), it is also possible to remove the remaining surfactant through filtration and washing, and then filter again to recover the microspheres. The washing step to remove the remaining surfactant can be typically performed using water, and the washing step can be repeated several times.

After the above step (d), the microspheres obtained can be dried using a conventional drying method to obtain the final dried microspheres.

Hereinafter, the present invention will be described in detail based on the following examples. However, the following examples are provided only to illustrate the present invention and the scope of the present invention is not limited thereto.

### Comparative Example 1: Manufacturing of Liquid Formulation of Semaglutide

A solvent was prepared by dissolving 4 mg of citric anhydride, 5.08 mg of sodium hydrogen phosphate dihydrate, 4 mg of polysorbate 20, 90 mg of carboxymethyl cellulose, 24 mg of sodium chloride, and 2.16 mg of sodium hydroxide in 4 ml of water. 0.25 mg of semaglutide was dissolved in the solvent.

### Comparative Example 2: Manufacturing of Semaglutide Microspheres

### <Manufacturing of Continuous Phase>

900 ml of distilled water, 4.5 g of PVA, and 4.5 g of NaCl were weighed and added to the reactor. A 0.5% (w/v) PVA aqueous solution was prepared by stirring at 1,000 rpm using a homogenizer (Overhead (IKA)).

### <Manufacturing of Dispersed Phase>

0.144 g of semaglutide and 2.4 ml of distilled water were weighed and added to a 20 ml vial, and the mixture was stirred at 150 rpm to prepare Solution A.

2.88 g of PLGA (intrinsic viscosity 0.1 dL/g to 1.9 dL/g) was added to 9.6 ml of dichloromethane, and the mixture was stirred at 300 rpm to prepare Solution B.

### <Emulsification>

Among the dispersed phases prepared above, the solution B was taken with a syringe and added to the solution A, and then stirred at 20,000 rpm for 1 minute using a homogenizer (IKA).

### <Recovering Microspheres>

After the above homogenization process was completed, the solution was immediately taken into a syringe and placed into a 1 L reactor in which the continuous phase was previously added for 60 seconds while stirring at 3,500 rpm using a homogenizer. Afterwards, the microspheres were solidified for a total of 20 hours, 5 hours at 25° C. and 15 hours at 35° C. After solidification was completed, the microspheres were obtained by filtering through a 5 μm filter. The obtained microspheres were then freeze-dried in a freeze dryer for more than 20 hours.

### Example 1: Manufacturing of Semaglutide Complex Injection Formulation

A solvent was prepared by dissolving 4 mg of citric anhydride, 5.08 mg of sodium hydrogen phosphate dihydrate, 4 mg of polysorbate 20, 90 mg of carboxymethyl cellulose, 24 mg of sodium chloride, and 2.16 mg of sodium hydroxide in 4 ml of water.

0.25 mg of semaglutide was weighed, and the microspheres manufactured in the above Comparative Example 2 were weighed so that the dosage of semaglutide would be 3 mg. Then, the solvent was added to these active ingredients and mixed to prepare a semaglutide complex injection formulation.

### Test Example 1: In-Vivo Pharmacokinetic Test Using Rats

To measure the blood semaglutide concentration,
the liquid formulation prepared in Comparative Example 1 was weighed so that the dosage of semaglutide was 0.25 mg/kg, and a liquid formulation injection was prepared.

The microspheres manufactured in Comparative Example 2 were weighed so that the dosage of semaglutide would be 3 mg/kg, and the microsphere formulation injection was prepared by dispersing them in a solvent prepared by dissolving 4 mg of citric anhydride, 5.08 mg of sodium hydrogen phosphate dihydrate, 4 mg of polysorbate 20, 90 mg of carboxymethylcellulose, 24 mg of sodium chloride, and 2.16 mg of sodium hydroxide in 4 ml of water.

The complex formulation manufactured in Example 1 was weighed so that the dosage of semaglutide would be 3.25 mg/kg, and a complex formulation injection was prepared.

The above semaglutide liquid formulation injection (Comparative Example 1), semaglutide microsphere formulation injection (Comparative Example 2), and complex formulation injection (Example 1) were administered to SD rats (Sprague-Dawley rats), respectively, and the blood semaglutide concentration was measured.

Specifically, each sample was subcutaneously injected into SD rats using a 23-gauge syringe, and approximately 0.2 ml of blood was collected at designated times, and the blood concentration of semaglutide was measured using LC-MS/MS. The area under the curve (AUC) was calculated from the blood concentration-time graph of semaglutide, and the bioavailability (%) was calculated using the following mathematical formula and is shown in Table 1 below.

$$Bioavailability ( \% ) = 100 ( \% ) \times \{ ( Complex formulation Blood ( drug ) concentration AUC ) \times ( Liquid formulation dosage ) \} / \{ ( Liquid formulation Blood ( drug ) concentration AUC ) \times ( Complex formulation dosage ) \} \qquad [ Mathematical Formula ]$$

TABLE 1

| | Comparative Example 2 | Example 1 |
|---|---|---|
| Dosage (mg/kg) | 3 | 3.25 |
| AUClast (ng * hr/mL) | 14,091.85 ± 3,664.91 | 29,760.80 ± 7,544.80 |
| Bioavailability (%) | 12.18 | 23.75 |

From Table 1 above, it is confirmed that the complex formulation injection (Example 1) has an increased dosage of about 8.3% compared to the microsphere formulation injection (Comparative Example 2), but the bioavailability is increased by about 95%. Therefore, these results indicate that the complex formulation of the present invention containing a GLP-1 receptor agonist provides significantly improved bioavailability.

### Comparative Example 3: Manufacturing of Tirzepatide Liquid Formulation

A solvent was prepared by dissolving 1 mg of citric anhydride, 1.27 mg of sodium hydrogen phosphate dihydrate, 1 mg of polysorbate 20, 22.5 mg of carboxymethylcellulose, 6 mg of sodium chloride, and 0.54 mg of sodium hydroxide in 1 ml of water. 0.06 mg of tirzepatide was dissolved in the solvent.

### Comparative Example 4: Manufacturing of Tirzepatide Microspheres

### <Manufacturing of Continuous Phase>

900 ml of distilled water, 4.5 g of PVA, and 4.5 g of NaCl were weighed and added to the reactor. A 0.5% (w/v) PVA aqueous solution was prepared by stirring at 1,000 rpm using a homogenizer (Overhead (IKA)).

### <Manufacturing of Dispersed Phase>

0.3 g of tirzepatide and 1.0 ml of distilled water were weighed and added to a 20 ml vial, and the mixture was stirred at 150 rpm to prepare Solution A.

1.2 g of PLGA (intrinsic viscosity 0.1 db/g to 1.9 dL/g) was added to 3 ml of dichloromethane, and the mixture was stirred at 300 rpm to prepare Solution B.

### <Emulsification>

Among the dispersed phases prepared above, the solution B was taken with a syringe and added to the solution A, and then stirred at 20,000 rpm for 1 minute using a homogenizer (IKA).

### <Recovering Microspheres>

After the homogenization process was completed, the solution was immediately taken into a syringe and placed into a 1 L reactor in which the continuous phase was previously added for 60 seconds while stirring at 7,000 rpm using a homogenizer. Afterwards, the microspheres were solidified for a total of 20 hours, 5 hours at 25° C. and 15 hours at 35° C. After solidification was completed, the microspheres were obtained by filtering through a 5 μm filter. The obtained microspheres were then freeze-dried in a freeze dryer for more than 20 hours.

### Example 2: Manufacturing of Tirzepatide Complex Injection Formulation

A solvent was prepared by dissolving 1 mg of citric anhydride, 1.27 mg of sodium hydrogen phosphate dihydrate, 1 mg of polysorbate 20, 22.5 mg of carboxymethylcellulose, 6 mg of sodium chloride, and 0.54 mg of sodium hydroxide in 1 ml of water.

0.06 mg of tirzepatide was weighed, and the microspheres manufactured in the above Comparative Example 4 were weighed so that the dosage of tirzepatide would be 3 mg, and then the solvent was added to these active ingredients and mixed to prepare a tirzepatide complex injection formulation.

### Test Example 2: In-Vivo Pharmacokinetic Test Using Beagles

To measure the blood tirzepatide concentration,
the liquid formulation prepared in Comparative Example 3 was weighed so that the dosage of tirzepatide was 0.06 mg/kg.

The microspheres manufactured in Comparative Example 4 were weighed so that the dosage of tirzepatide would be 3 mg/kg, and the microsphere formulation injection was prepared by dispersing them in a solvent prepared by dissolving 1 mg of citric anhydride, 1.27 mg of sodium hydrogen phosphate dihydrate, 1 mg of polysorbate 20, 22.5 mg of carboxymethylcellulose, 6 mg of sodium chloride, and 0.54 mg of sodium hydroxide dissolved in 1 ml of water.

The complex formulation manufactured in the above Example 2 was weighed so that the dosage of tirzepatide was 3.06 mg/kg, and a complex formulation injection was prepared.

The tirzepatide liquid formulation injection (Comparative Example 3), tirzepatide microsphere formulation injection (Comparative Example 4), and complex formulation injection (Example 2) were administered to beagles, and then the blood tirzepatide concentration was measured.

Specifically, each sample was injected subcutaneously into a beagle using a 23-gauge syringe, blood was collected at designated times, and the blood concentration of tirzepatide was measured using LC-MS/MS. The area under the curve (AUC) was calculated from the blood concentration-time graph of tirzepatide, and the bioavailability (%) was calculated using the following mathematical formula and is shown in Table 2 below.

$$Bioavailability ( \% ) = 100 ( \% ) \times [ ( Complex formulation Blood ( drug ) concentration AUC ) \times ( Liquid formulation dosage ) \} / \{ ( Liquid formulation Blood ( drug ) concentration AUC ) \times ( Complex formulation dosage ) \} \qquad [ Mathematical Formula ]$$

TABLE 2

| | Comparative Example 4 | Example 2 |
|---|---|---|
| Dosage (mg/kg) | 3 | 3.06 |
| AUClast (ng * hr/mL) | 852,482,53 ± | 1,035,296,82 ± |
| | 264,582.42 | 315,801.28 |
| Bioavailability (%) | 24.85 | 29.58 |

From Table 2 above, it is confirmed that the complex formulation injection (Example 2) has an increased dosage of about 2% compared to the microsphere formulation injection (Comparative Example 4), but the bioavailability is increased by about 198. Therefore, these results indicate that the complex formulation of the present invention containing a GIP/GLP-1 receptor dual agonist provides significantly improved bioavailability.

1. A pharmaceutical composition, comprising:
as a first active ingredient, at least one selected from the group consisting of a GLP-1 receptor agonist, a GIP/GLP-1 receptor dual agonist, and a GLP-1/GIP/GCG receptor triple agonist, or a pharmaceutically acceptable salt thereof,
as a second active ingredient, a microsphere containing at least one selected from the group consisting of a GLP-1 receptor agonist, a GIP/GLP-1 receptor dual agonist, and a GLP-1/GIP/GCG receptor triple agonist, or a pharmaceutically acceptable salt thereof, and
a pharmaceutically acceptable liquid carrier.

2. The pharmaceutical composition of claim 1, wherein the second active ingredient included in the microsphere is contained in an amount of 3 to 1000 times the weight of the first active ingredient.

3. The pharmaceutical composition of claim 1, wherein the first active ingredient and the second active ingredient are contained in a total amount of 0.001 mg to 2,000 mg based on 1 mL of the liquid carrier.

4. The pharmaceutical composition of claim 1, wherein the liquid carrier further comprises at least one selected from the group consisting of water, surfactant and polyhydric alcohol and a pharmaceutically acceptable excipient.

5. The pharmaceutical composition of claim 4, wherein the surfactant is at least one selected from the group consisting of Poloxamer 188, Poloxamer 407, Polysorbate 20, Polysorbate 60, Polysorbate 80, Polyoxyethylene oleyl ether, Polyoxyethylene cetyl ether, Polyoxyethylene stearyl ether, and Polyoxyethylene lauryl ether.

6. The pharmaceutical composition of claim 4, wherein the polyhydric alcohol is at least one selected from the group consisting of ethylene glycol, propylene glycol, polyethylene glycol, glycerol, erythritol, threitol, xylitol, ribitol, mannitol, sorbitol, and maltitol.

7. The pharmaceutical composition of claim 4, wherein the excipient is at least one selected from the group consisting of a buffer, an isotonic agent, a preservative, and a pH regulator.

8. The pharmaceutical composition of claim 1, wherein at least one selected from the group consisting of a GLP-1 receptor agonist, a GIP/GLP-1 receptor dual agonist and a GLP-1/GIP/GCG receptor triple agonist of the first active ingredient or the second active ingredient is at least one selected from the group consisting of exenatide, liraglutide, lixisenatide, albiglutide, dulaglutide, semaglutide, tirzepatide, cotadutide, taspoglutide, and retatrutide.

9. The pharmaceutical composition of claim 1, wherein the microsphere formulation further comprises a biodegradable polymer.

10. The pharmaceutical composition of claim 9, wherein the biodegradable polymer is at least one selected from polylactic acid, polylactide, polylactic-co-glycolic acid, polylactide-co-glycolide (PLGA), polyphosphazine, polyiminocarbonate, polyphosphoester, polyanhydride, polyorthoester, copolymer of lactic acid and caprolactone, polycaprolactone, polyhydroxyvalerate, polyhydroxybutyrate, polyamino acid, and copolymer of lactic acid and amino acid.

11. The pharmaceutical composition of claim 9, wherein the second active ingredient and the biodegradable polymer contained in the microsphere formulation are included in a weight ratio of 1:1 to 50.

12. The pharmaceutical composition of claim 1, wherein the release rate of the second active ingredient of the microsphere within 7 days is 60 wt % or less.

13. The pharmaceutical composition of claim 1, wherein the pharmaceutical composition is in the form of an injection.

14. The pharmaceutical composition of claim 1, wherein the pharmaceutical composition is administered to the subject at intervals of 1 to 6 months.

15. A pharmaceutical composition, which is the pharmaceutical composition of claim 1, and used for the prevention or treatment of diabetes, hypertension, hyperlipidemia, obesity, non-alcoholic steatohepatitis, metabolic steatohepatitis, cardiovascular disease, or degenerative neurodegenerative disease.
